(19) United States
(12) Patent Application Publication
(10) Pub. No.: US 2005/0186554 A1
(43) Pub. Date: Aug. 25, 2005

(54) Image analysis and assay system

(76) Inventors: Vladimir Temov, Los Gatos, CA (US); Ilya Ravkin, Palo Alto, CA (US)

Correspondence Address: KOLISCH HARTWELL, P.C., 520 S.W. YAMHILL STREET, SUITE 200, PORTLAND, OR, 97204, US

(21) Appl. No.: 11/039,077

(22) Filed: Jan. 18, 2005

Related U.S. Application Data
(60) Provisional application No. 60/537,454, filed on Jan. 15, 2004.

(51) Int. Cl. C12Q 1/00; G06F 19/00; G01N 33/48; G01N 33/50

(52) U.S. Cl. 435/4; 702/19

(57) ABSTRACT

Systems for determining and/or analyzing the distribution and dynamics of cellular components.

### CROSS-REFERENCES TO PRIORITY APPLICATIONS

This application is based upon and claims the benefit under 35 U.S.C. § 119(e) of the following U.S. provisional patent applications, which are incorporated herein by reference in their entirety for all purposes: Ser. No. 60/537,454, filed Jan. 15, 2004; and Ser. No. ______, filed Jan. 17, 2005, titled IMAGE ANALYSIS SYSTEM, and naming Vladimir Temov and llya Ravkin as inventors.

### CROSS-REFERENCES TO RELATED APPLICATIONS

This application incorporates by reference in their entirety for all purposes the following U.S. patent applications: Ser. No. 09/549,970, filed Apr. 14, 2000; Ser. No. 09/694,077, filed Oct. 19, 2000; Ser. No. 10/120,900, filed Apr. 10, 2002; Ser. No. 10/238,914, filed Sep. 9, 2002; Ser. No. 10/273,605, filed Oct. 18, 2002; Ser. No. 10/282,904, filed Oct. 28, 2002; Ser. No. 10/282,940, filed Oct. 28, 2002; Ser. No. 10/382,796, filed Mar. 5, 2003; Ser. No. 10/382,797, filed Mar. 5, 2003; Ser. No. 10/382,818, filed Mar. 5, 2003; Ser. No. 10/407,630, filed Apr. 4, 2003; Ser. No. 10/444,573, filed May 23, 2003; Ser. No. 10/445,291, filed May 23, 2003; Ser. No. 10/713,866, filed Nov. 14, 2003; Ser. No. 10/842,954, filed May 10, 2004; Ser. No. 10/901,942, filed Jul. 28, 2004; and Ser. No. 10/942,322, filed Sep. 15, 2004.

This application also incorporates by reference in their entirety for all purposes the following U.S. provisional patent applications: Ser. No. 60/129,664, filed Apr. 15, 1999; Ser. No. 60/170,947, filed Dec. 15, 1999; Ser. No. 60/241,714, filed Oct. 18, 2000; Ser. No. 60/259,416, filed Dec. 28, 2000; Ser. No. 60/293,863, filed May 24, 2001; Ser. No. 60/299,267, filed Jun. 18, 2001; Ser. No. 60/299,810, filed Jun. 20, 2001; Ser. No. 60/307,649, filed Jul. 24, 2001; Ser. No. 60/307,650, filed Jul. 24, 2001; Ser. No. 60/310,540, filed Aug. 6, 2001; Ser. No. 60/317,409, filed Sep. 4, 2001; Ser. No. 60/318,156, filed Sep. 7, 2001; Ser. No. 60/328,614, filed Oct. 10, 2001; Ser. No. 60/343,682, filed Oct. 26, 2001; Ser. No. 60/343,685, filed Oct. 26, 2001; Ser. No. 60/344,482, filed Oct. 26, 2001; Ser. No. 60/344,483, filed Oct. 26, 2001; Ser. No. 60/345,606, filed Oct. 26, 2001; Ser. No. 60/348,025, filed Oct. 26, 2001; Ser. No. 60/348,027, filed Oct. 26, 2001; Ser. No. 60/359,207, filed Feb. 21, 2002; Ser. No. 60/362,001, filed Mar. 5, 2002; Ser. No. 60/362,055, filed Mar. 5, 2002; Ser. No. 60/362,238, filed Mar. 5, 2002; Ser. No. 60/370,313, filed Apr. 4, 2002; Ser. No. 60/383,091, filed May 23, 2002; Ser. No. 60/383,092, filed May 23, 2002; Ser. No. 60/413,407, filed Sep. 24, 2002; Ser. No. 60/413,675, filed Sep. 24, 2002; Ser. No. 60/421,280, filed Oct. 25, 2002; Ser. No. 60/426,633, filed Nov. 14, 2002; Ser. No. 60/469,508, filed May 8, 2003; Ser. No. 60/473,064, filed May 22, 2003; Ser. No. 60/503,406, filed Sep. 15, 2003; Ser. No. 60/523,747, filed Nov. 19, 2003; and Ser. No. 60/585,150, filed Jul. 2, 2004.

This application incorporates by reference in their entirety for all purposes the following PCT patent application: Serial No. PCT/US01/51413, filed Oct. 18, 2001, and published as Pub. No. WO 02/37944 on May 16, 2002.

### INTRODUCTION

The organization and dynamics of molecules and supramolecular assemblies plays an important role in the function of cellular systems. Eucaryotic cells, in particular, are highly organized, with many structurally and/or functionally related components organized into specific locations or compartments such as organelles. For example, selected cellular components associated with energy production in eucaryotic cells are organized into mitochondria, while selected cellular components associated with cellular control and inheritance are organized into the nucleus. Eucaryotic cells, more generally, may include a number of different organelles or compartments, organized for a number of different functions, including the nucleus, mitochondria, chloroplasts, lysosomes, peroxisomes, vacuoles, Golgi apparatus, rough and smooth endoplasmic reticulum, centrioles, plasma membrane, nuclear envelope, endosomes, secretory vesicles, and so on.

The components of these different compartments, and of cells and biological organisms in general, may be highly dynamic. Thus, specific molecules may diffuse and/or be actively transported between different regions in the cell and/or between the cell and the extracellular medium. In some cases, molecules may move, or translocate, from one compartment to another, in response to changes in cell cycle, cell signaling (e.g., hormones), disease state, and so on. Moreover, in the case of molecules such as enzymes, the mechanisms that control such distribution and dynamics may be independent of the mechanisms that control or effect catalysis, meaning that they may provide unique, previously unexploited targets for candidate drugs, potentially allowing compounds with similar functionalities (such as kinases) to be targeted based on dissimilar localization or translocalization signals or behavior. Significantly, many molecules potentially associated with disease states, such as transcription factors and kinases, translocate, particularly from cytoplasm to nucleus, in the course of the activation process.

The “natural” approach in image analysis, such as translocation image analysis, is to segment the image into compartments such as nuclei and cytoplasm of individual cells, measure the amount of signal stain in each, and calculate a measure of translocation as the difference or the ratio of the two [1,2]. A variation on this approach is to analyze signal stain in smaller compartments defined by their spatial relation to the center or the boundary of the nucleus [3,4]. In all cases, these methods require image segmentation. Thus, because segmentation usually is sensitive to image peculiarities and artifacts, and further may not scale well with magnification, there is a need for systems that do not require, or at least do not critically depend, on segmentation.

### SUMMARY

The present teachings provide systems for determining and/or analyzing the distribution and dynamics of cellular components.

### BRIEF DESCRIPTION OF THE DRAWINGS

FIG. 1 is a schematic view of a general framework for image analysis, in accordance with the present teachings. Panel A shows cells with different reporter images, Panel B shows an exemplary three-dimensional histogram, and Panel C shows three exemplary two-dimensional histograms.

FIG. 2 is a series of micrographs showing the nuclear translocation of NFkB in MCF7 cells. Left-negative, middle-intermediate, right-positive states. Images of FITC stain acquired with a 10× objective.

FIG. 3 is a set of exemplary stain and counterstain profiles through model and real cells. A,B—model; C,D—real; A,C—negative; B,D—positive. S—signal stain; CS—counterstain.

FIG. 4 is a set of cross-histograms of signal stain (vertical axis) and counterstain (horizontal axis) in an ideal model system (A,B), perturbed model system (C,D), and a real cell (E,F). The scale on both axes is 0-255. The lines show the calculated approximation.

FIG. 5 is a set of graphs showing the approximation of slope (left) as a function of counterstain intensity in subsets of distribution (right) increasing from right to left. The middle lines show the resulting value of the slope. Top panel—nuclear localization of protein; bottom panel—cytoplasm localization of protein.

FIG. 6 is a flow chart of possible computational scenarios for image analysis of a cytoplasm-to-nucleus translocation assay.

FIG. 7 is a set of graphs showing individual (cell-by-cell) and global slopes at different magnifications.

FIG. 8 is a set of graphs showing how nucleoli change the two-dimensional stain distribution by creating a cluster of points, shown by the dotted oval, that lead to underestimation of the slope. This effect may be corrected by filling the holes in the signal image corresponding to the nucleoli. A,B—analysis of a single cell; C,D—global analysis of many cells. A,C—uncorrected data; B,D—corrected (filled) data. Slopes: (A) 61, (B) 72, (C) 1.21, (D) 1.61.

FIG. 9 is a series of panels showing an exemplary method for filling nucleoli, in accordance with aspects of the present teachings.

FIG. 10 is a set of histograms of slope distributions in a dose-dependent set of images of cytoplasm to nucleus translocation of NFκB in MCF7 cells. The histograms show the percentage of cells with a given slope (vertical axis) versus slope (horizontal axis).

FIG. 11 is a bar graph showing variance of the data set explained by first principal components of the slope histogram.

FIG. 12 is a graph showing weights of the principal components on the original features bins in the slope histogram.

FIG. 13 is a plot showing distribution of images of cytoplasm-to-nucleus translocation assay in the space of the first two principal components of the slope histogram. The dotted arrow shows the increase in the dose of TNFA.

FIG. 14 is a nuclear translocation dose curve, showing average cell slope (vertical axis) versus TNFα concentration (horizontal axis). V-factor=0.77.

FIG. 15 is a graph of V-factors for nuclear translocation measure (vertical axis) as a function of interpolated magnification (horizontal axis) at different image sizes.

FIG. 16 is a series of micrographs showing membrane-to-cytoplasm translocation. Left-negative, middle-intermediate, right-positive states.

FIG. 17 is a set of graphs for the joint distribution of nuclear counterstain and signal stain in a model system for membrane-to-cytoplasm translocation. Top—membrane localization, bottom—cytoplasm localization. Left—two-dimensional distribution of stains, right—profiles through the model cell.

FIG. 18 is a set of graphs for the joint distribution of signal stain and counter stain in a real cell. Top—membrane localization, bottom—cytoplasm localization. Left—two-dimensional distribution of stains, right—profiles through the real cell.

FIG. 19 is a set of graphs for the joint distribution of signal stain and membrane counterstain in a model system perturbed with random noise for membrane-to-cytoplasm translocation. Top—membrane localization, bottom—cytoplasm localization. Left—two-dimensional distribution of stains, right—profiles through the model cell.

FIG. 20 is a series of images of a Transfluor® assay at objective magnification 10× with 2*2 binning. A—negative, B—intermediate, C—positive.

FIG. 21 is a set of brightness profiles through cells, for the assay of FIG. 24, in an original image, in the image opened by a structuring element of size 1, and in the image opened by a structuring element of size 4. Left—negative, middle—intermediate, right—positive states.

FIG. 22 is a set of curves showing the granular spectrum for negative, intermediate, and positive states of the Transfluor assay. Horizontal axis—size of opening, vertical axis—fraction of the image volume at this opening.

FIG. 23 is a set of curves showing dependency of z-value (vertical axis) for relative granularity on magnification (horizontal axis) and image size (as noted in figure). The range of best assay performance is outlined.

### DETAILED DESCRIPTION

The present teachings provide systems for determining and/or analyzing the distribution and dynamics of cellular components. These systems, which may include apparatus, methods, compositions, and kits, for preparing, positioning, treating, and/or analyzing samples, among others, may be particularly suitable for use in studies of joint distributions of two or more substances, particularly where one or more of these substances function as reference or counter stains, and one or more of these substances function as signal stains. For example, in some embodiments, the reference or counter stain(s) may be used as a marker for cellular features or compartments, and the signal stain(s) may be used to study of the distribution of a substance capable of translocation with the cell. Such translocation may include cytoplasm-to-nucleus translocation, nucleus-to-cytoplasm translocation, membrane-to-cytoplasm (or nucleus) translocation, cytoplasm (or nucleus)-to-membrane translocation, and so on.

Preparing samples, as used here, may include, among others, (1) selecting, separating, enriching, growing, modifying, and/or synthesizing a composition, a cellular component, a cell, a tissue, and/or any other assay component, among others, (2) selecting, forming, and/or modifying sample carriers and/or sample containers, such as coded carriers and/or multiwell systems, such as microplates, respectively, and/or (3) associating samples and sample carriers, and/or samples and sample containers, and so on.

Positioning samples, as used here, may include positioning the samples (and/or any associated sample carriers) for treatment and/or analysis, among others. Such positioning may include, among others, (1) mixing samples, (2) dispensing samples at treatment and/or analysis sites, and/or (3) dispersing samples at treatment and/or analysis sites, for example, to allow access to the samples and/or visualization of the samples, respectively.

Treating samples, as used here, may include exposing the samples to some condition, such as a chemical, a temperature, a concentration (e.g., an ion concentration, such as hydrogen ion (pH), salt ion, etc.), and/or the like, and/or a change thereof. These conditions may comprise a candidate modulator, for example, a condition of unknown or partially characterized effect, such as a candidate transcription modulator.

Analyzing samples, as used here, may include observing and/or measuring, qualitatively and/or quantitatively, a condition of the sample (e.g., size, mass, identity, etc.,) and/or a condition caused by the sample (e.g., depletion of an enzyme substrate, production of an enzyme product, etc.), using any suitable method(s) (e.g., optical (imaging, absorption, scattering, luminescence, photoluminescence (e.g., fluorescence or phosphorescence), chemiluminescence, etc.), magnetic resonance, and/or hydrodynamics, among others). Such analyzing further may include detecting and/or interpreting a presence, amount, and/or activity of the sample, or a modulator thereof, including agonists and/or antagonists, and/or determining trends or motifs from the analysis of multiple samples. Such analyzing further may include determining and/or analyzing the joint distribution of two or more stains or other indicators of location and/or activity in biological systems, for example, for use in translocation assays, among others.

The systems provided by the present teachings further include but are not limited to those described below in the Examples, and may be combined, optionally, with apparatus, methods (including labeling and transfection methods), compositions (including molecules, cells, tissues, and the like), and/or kits, or components thereof, described in the various patent applications listed above under Cross-References and incorporated herein by reference.

### EXAMPLES

The following examples describe selected aspects and embodiments of the present teachings, particularly exemplary distribution and dynamics assays. These examples are included for illustration and are not intended to limit or define the entire scope of the present teachings. Further aspects of the present teachings are described in the various patent applications listed above under Cross-References and incorporated herein by reference, particularly U.S. Provisional Patent Application Ser. No. 60/537,454, filed Jan. 15, 2004; and U.S. Provisional patent application Ser. No. ______, filed Jan. 17, 2005, titled IMAGE ANALYSIS SYSTEM, and naming Vladimir Temov and llya Ravkin as inventors. These two provisional patent applications include color drawings and additional text that complement and further illustrate the concepts described below, particularly in Examples 1, 2, and 4.

### Example 1

### Cytoplasm to Nucleus Translocation Assay

1.1. Background

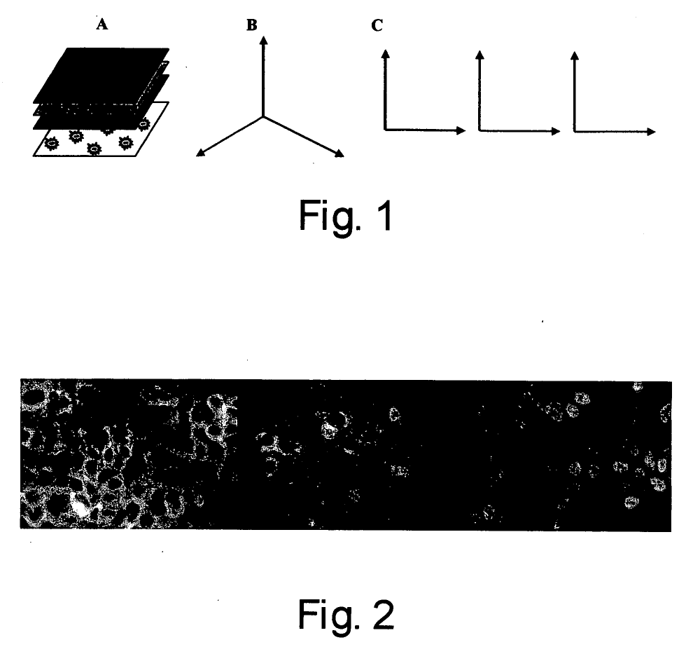

FIG. 1 shows the general data framework for an exemplary embodiment of the present teachings: a cytoplasm-to-nucleus (or nucleus-to-cytoplasm) translocation assay. A field of view is digitally acquired (or acquired in analog, and converted to digital) at different spectral regions and/or with different optical modalities, so that there is, or can be made to be, pixel-to-pixel correspondence among all images from the same field. The different spectral regions can include different wavelength bands, such as blue and green, among others. The different optical modalities can include different imaging techniques, such as photoluminescence and transmission, among others. The framework allows analysis of two-dimensional distributions, or series of such distributions, or higher-dimensional distributions, up to the number of reporter images. Such joint distributions may be analyzed in different subsets of pixels, ranging from the whole image down to portions of individual cells.

1.2 Method Based on 2D Distribution of Stains. Model and Experimental Distributions.

The present teachings may include analysis of translocation events based on the joint distribution of signal and counter-stains. Representative data were collected and analyzed for the translocation of the transcription factor NFκB in MCF7 cells in response to TNFα concentration (see, e.g., FIG. 2). To find a robust measure of nuclear translocation, we also have defined and studied a model of the spatial distribution of the nuclear counterstain and of the signal stain as it moves from the cytoplasm to the nucleus. The model results, which were compared with the experimental data, were studied under various conditions and perturbations to find measures that are robust.

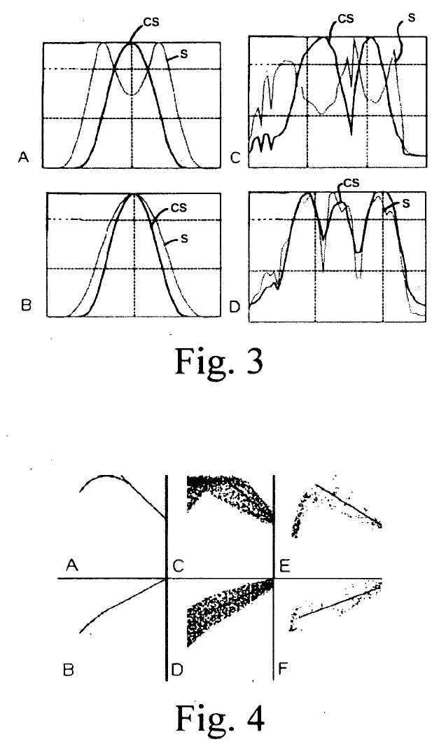

FIG. 3 shows intensity profiles along a line drawn through model (Panels A and B) and real (Panels C and D) cells containing a signal stain and a counterstain. The model cells include a bell-shaped counterstain (nuclear) intensity distribution, and either (Panel A) a wider bell-shaped signal stain intensity distribution, with a bell-shaped crater, corresponding to a negative correlation between signal and counter stains, or (Panel B) a bell-shaped signal stain intensity distribution, corresponding to a positive correlation between signal and counter stains. The real cells show substantially similar profiles as the model cells. Here, the profile in Panel C shows a negative correlation, plotted through two cells, and the profile in Panel D shows a positive correlation, plotted through three cells. All profiles, model and real, are normalized independently to their respective intensity maxima.

1.3 Quantification of Cross-Correlations

The joint distributions of, or cross-correlations between, signal stain(s) and counterstain(s), and/or changes thereof, may be observed and/or analyzed using any suitable method(s). In some cases, it may be possible and sufficient simply to observe a value or change visually. However, in most cases, it will be desirable or necessary to observe values or changes quantitatively, particularly in contexts such as screening that may involve analysis of many samples.

FIG. 4 shows cross-histograms, or correlation plots, of signal stain (vertical axis) and counterstain (horizontal axis). Specifically, the intensity of the signal stain (or some suitable measure or function thereof) is plotted as a function of the associated intensity of the counterstain (or some suitable measure or function thereof). Thus, data points in the lower left quadrant of the plot correspond to portions of the image having low concentrations of both signal stain and counterstain, data points in the upper right quadrant correspond to regions of the image having high concentrations of both signal stain and counterstain, data points in the upper left quadrant correspond to regions of the image having high concentrations of signal stain but low concentrations of counterstain, and data points in the lower right quadrant correspond to regions of the image having low concentrations of signal stain but high concentrations of counterstain. In these plots, negative correlations will tend to show up as distributions of data points having negative slopes, and positive correlations will tend to show up as distributions of data points having positive slopes. To derive stable measures that characterize transitions from the negative to the positive case, or vice versa, we analyzed joint distributions of the stains on the model and real cells. In the ideal case, the model spatial stain distributions are circularly symmetrical and aligned, as shown in FIG. 3(A,B). The cross-histograms for these cases are shown in FIG. 4(A,B). If the model is perturbed by offsetting the centers of the two stains, by changing shape from circular to oval, and/or by adding noise, among others, the distributions become fuzzy, as shown in FIG. 4(C,D). Typical negative and positive real cells have cross-histograms as shown in FIG. 4(E,F). These distributions suggest that a suitable translocation measure can be defined as the slope (or more crudely the sign of the slope) of a straight-line segment approximating the right side of the cross-histogram. This portion of the distribution corresponds to the more intense nuclear staining and also is close to the center of the nucleus. The farther from the center, the more diffuse the distribution, and the less reliable the approximation.

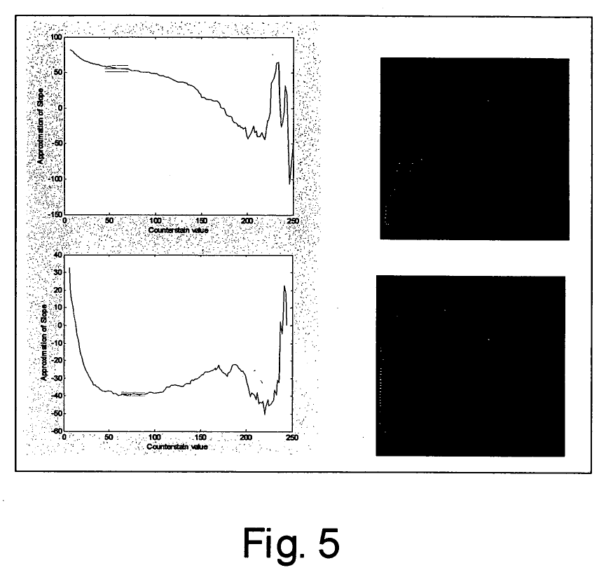

FIG. 5 shows an approximation of slope (left) as a function of counterstain intensity in subsets of distributions (right) increasing from right to left. The portion of the distribution that is used for approximation with the straight line is found by plotting the approximated slope going from right to left and selecting the range where this approximation is the most stable.

FIG. 5 also shows a possible variation on this method. This variation may involve calculating two more slopes. The top line is the regression line calculated on all points above the original slope segment (which we will refer to as slope1); the bottom line is the regression line calculated on all points below the original slope segment (which we will refer to as slope2). If all three slopes (i.e., the original slope, slope1, and slope2) have the same sign, then the result is the one with the greatest absolute value. However, if they have different signs, then the original slope is chosen. We call this measure slope3.

1.4 Global vs. Cell-By-Cell vs. Cluster-By-Cluster Analysis

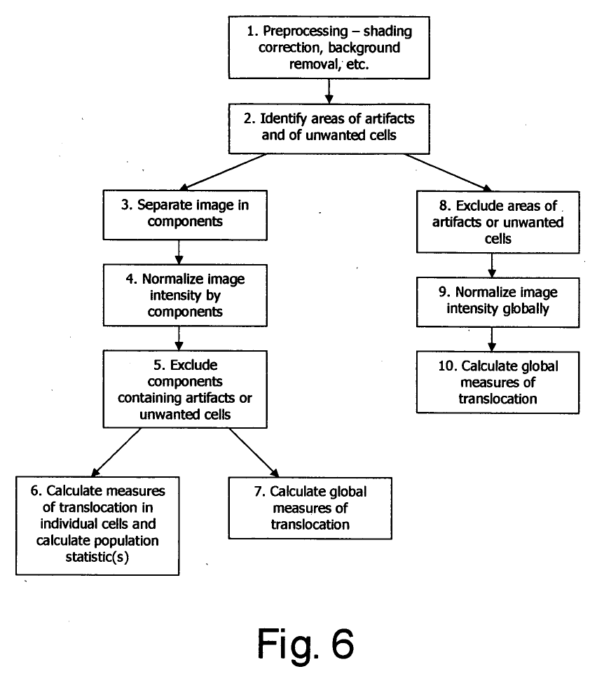

The present teachings can be applied to entire images, or portions thereof, including but not limited to selected portions of individual cells, selected cells, and/or selected clusters or regions of cells, among others. FIG. 6 shows a flow chart of possible computation scenarios for image analysis in cytoplasm-to-nucleus translocation assays.

Application of the method on the individual cell level may offset or neutralize variations in expression or staining, which in the case of translocation may be not informative. In some cases (e.g., low magnification), partitioning the image into individual cells is difficult; then the analysis can be done on clusters of closely situated cells. This may not account for biological variation among the cells in the cluster, but it will account for variation among clusters. The variation among clusters also can be due to technical or experimental reasons, such as nonuniformity in illumination. The analysis may be applied to individual cells, without knowledge of the cell or nuclear boundary, but simply with knowledge of the area within which a separate cell is contained.

Global analysis has its advantages too. It may be faster and/or more stable at low magnification. The objections to global (whole well) analysis usually are that it does not account for variation among cells and that it does not exclude unwanted cells. The second issue can be addressed directly, regardless of how the accepted cells are analyzed, individually or as a whole. For the purpose of this discussion, there are two issues: (1) global analysis may not give a measure of average response that is as good as individual cell analysis, and (2) average measure alone may not be sufficiently informative. The first issue may be overcome, at least partly, by normalizing intensity, in which case the global measures often are as good as averages of individual cell measures, see FIG. 7. The second issue is addressed in Section 1.9.

1.5 Partitioning into components. Markers. Watersheds of Combined Intensity Images.

Images may be analyzed as a whole and/or in portions or components. Partitioning into components may serve two purposes: (1) facilitating analysis of selected image features, such as cell clusters, individual cells, and/or portions thereof; and (2) facilitating, as a step in the procedure, optional intensity equalization.

Partitioning may be performed using any suitable mechanism(s), such as: (1) finding of markers, and (2) finding of separation lines.

Markers may be found by any suitable algorithm(s). For example, a fixed value (marker contrast) may be subtracted with saturation from the image of nuclear counterstain, and the resulting image reconstructed [11] within the image of nuclear counterstain. This image then may be subtracted from the counterstain image and converted to a binary image. The components of this binary image are the markers. A further restriction may be imposed on markers: only markers that have at least one pixel above a given threshold (marker brightness) are retained for the second step. Depending on magnification and noise level, the image of nuclear counterstain may be smoothed prior to this algorithm. This method of determining markers can handle cells of different size and shape. Other methods, e.g., based on top-hat transform [11], also may be used.

Separation lines between components (e.g., nuclei, cells, etc.) may be found by any suitable algorithm(s). For example, separation lines may be defined as the watershed [5,6,10] of the inverted image of the linear combination of the counterstain image and the signal stain image. The reason to use linear combination rather than just the nuclear counterstain image is that cells are often nonsymmetrical and unevenly spaced. Separation lines from a nuclear stain image may cut through the middle of cells. The use of signal stain produces more accurate separation lines. Coefficients of the linear combination may be varied depending on the peculiarities of staining and image acquisition.

1.6 Normalization of Intensities

The joint distributions of counterstain and signal stain may be normalized to their respective maxima. This can be done on the distribution or on the original image. The result is the same, but normalizing the image provides additional feedback for the user and may reveal features that were not seen before normalization.

Normalization (and/or other resealing) can be performed on entire images, and/or portions thereof, using any suitable mechanism(s). For example, normalization can be done in components, as described above. In this case, all pixels from a component are multiplied by the same number, separately for signal stain and for counterstain. Alternatively, normalization can be done without partitioning the image by fitting a smooth surface to the images of signal stain and counterstain. Normalization may have the effect of locally equalizing the image, and may involve resealing the image so that the maximum value and/or an integrated value equals unity or some other preselected value.

1.7 Artifact Removal. Gating. Classification.

Physiological variability and/or other conditions can create artifacts that affect assay results. For example, some cells, such as MCF7 cells at sufficiently low densities, have a noticeable percentage of mitotic cells in which the nuclear membrane has broken down and the chromosomes have condensed. These cells, whose chromosomes can stain intensely with a nucleic acid dye, may produce spurious “negative” results and upset the positive state of the assay. However, these cells can be excluded (or removed) on the basis of their high nuclear staining intensity and/or apparently undersized “nucleus,” among others. Here, “excluded” may include not being used in subsequent calculations and/or tabulations, and/or not being used in a final determination of assay results, among others. The information or results that may be excluded can include portions and/or the entireties of one or more cells, one or more regions of cells, and so on. Thus, in an exemplary embodiment in which cells are in contact with or over- or underlay a fluorescent filament, the affected portions of the cell(s) may be excluded, and/or all of the affected cell(s) may be excluded, among others. More generally, any artifact such as other cell types and/or non-cellular artifacts, that can be differentiated by its intensity, shape, size, and/or position, among others, also can be excluded.

Conversely, in some cases, cross-correlations, such as the value of the slope in a cross-histogram, can be used for classification of cells, rather than exclusion of cells. For example, a mitotic cell may give rise to a negative slope in a cross-histogram, since signal stain will tend to be excluded from counter (nuclear) stained regions, whereas an interphase cell may give rise to a positive slope, at least if there is a positive correlation between the locations of signal stain and counterstain.

1.8 Preprocessing of Images. Nucleoli Removal by Filling Holes.

Proteins and other molecules that translocate from cytoplasm to nucleus commonly do not enter the nucleoli. This tendency can create artifacts, unless taken into account, because it may be interpreted as a lack of translocation.

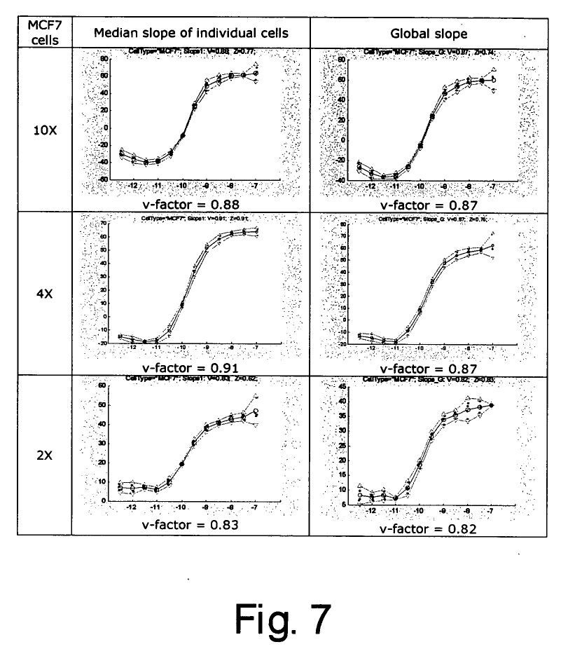

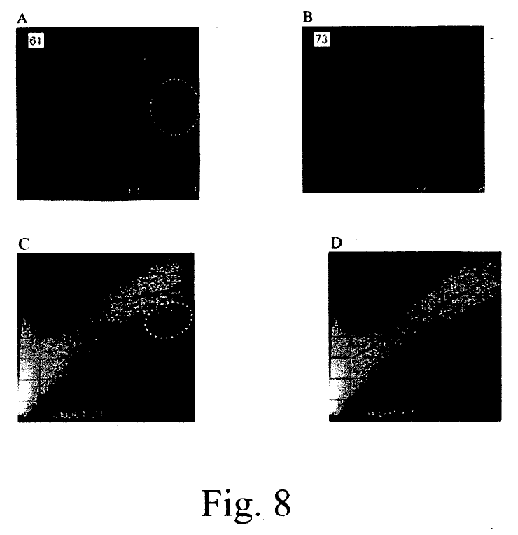

FIG. 8 shows errors in the estimation of cross-histogram slopes associated with nucleoli. These errors arise because regions with high counterstain (i.e., nuclear stain) intensity are associated with regions of low signal stain intensity, even in the presence of translocation, because the signal stain is excluded from the nucleoli.

These artifacts can be addressed by identifying nucleoli and excluding their mask from the nuclear mask. However, this approach suffers from the same drawbacks as segmentation, and masks, in general (see Background).

These artifacts also can be addressed by changing the image of the signal stain so that it does not have the undesired properties, for example, by filling the holes as if there were no nucleoli. A challenge is to fill nucleoli but not to fill whole nuclei of negative cells, which also look like holes. One approach is to (1) make an image of pixelwise multiplication of signal and counterstain images, (2) fill holes [10] in the image, and then (3) add the increment to the original signal stain image. This increment can be multiplied by a constant greater than 1. A drawback of this approach is that holes (nucleoli) that are close to the edge of the nucleus may not fill completely. An alternative approach is to fill holes on the signal stain image directly, but to select only those among them that fall into a size range that is characteristic of nucleoli (i.e., that is neither too small nor too large, for a given cell type, set of conditions, and so on).

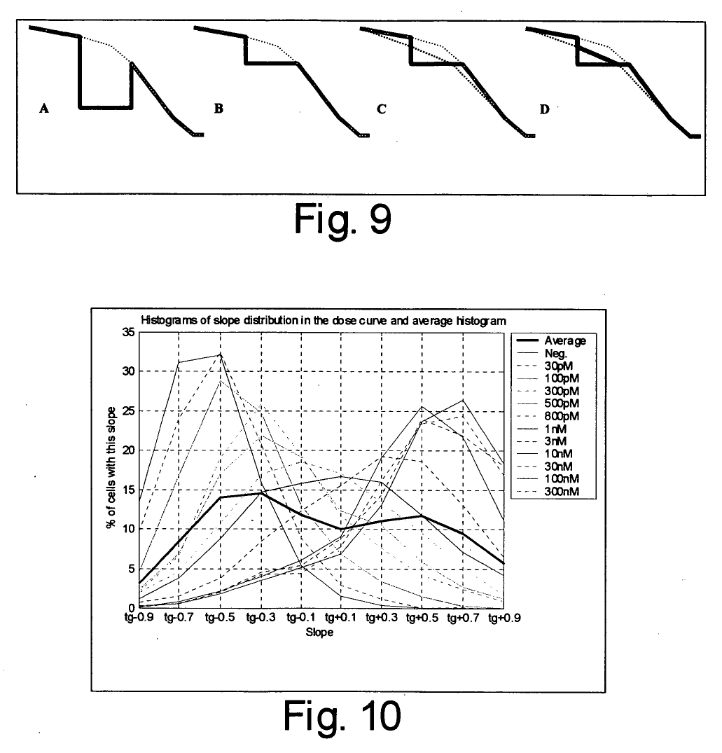

FIG. 9 shows exemplary steps for filling nucleoli. The dotted line in each Panel shows the imaginary profile of signal stain, in the absence of nucleoli. Panel A shows the intensity profile through a nucleolus. Panel B shows the intensity profile after filling the hole. Panel C shows the intensity profile after smoothing. Finally, Panel D shows the maximum of smoothed and filled images selected under a mask of filled areas. The optional smoothing steps may further improve the intensity distribution after filling.

Images may, more generally, be modified if this leads to a better estimate of the final assay measure of interest, for example, with quality measured as described in Section 1.10. One example is smoothing. This may, in some cases, improve slope measures, especially if the images are acquired on an instrument having shallow depth of field.

1.9 Heterogeneity. Population Measures of Position and Variation. Principal Component Analysis.

The present teachings include systems for addressing or interpreting heterogeneity in cell populations. For example, in the process of translocation of proteins from cytoplasm to nucleus, not all cells behave synchronously, and different cells may even exhibit opposite behaviors.

In some cases, it may be possible or desirable to find or determine a single (scalar) measure of translocation. In such cases, it may be reasonable to reduce the population to a positional measure, such as a mean (average), median, mode, etc. Measures of variation in the population of cells also may provide valuable information. In the example presented here, measures of variation, such as standard deviation, median deviation from median, etc., exhibit dose-related behavior, just like measures of position.

In the same and/or other cases, it may be possible or desirable to find or determine a multidimensional (vector) measure of translocation. In such cases, it may be reasonable to use a multidimensional statistical method, such as principal component analysis [12] (PCA). A multidimensional analysis may provide additional or more detailed information about cell behavior and heterogeneity.

FIG. 10 is a set of histograms or curves showing the distribution of slopes in a set of images showing the dose-dependent translocation of NFκB from cytoplasm to nucleus in MCF7 cells. Here, the percentage of cells with a given slope (vertical axis) is shown as a function of slope (horizontal axis).

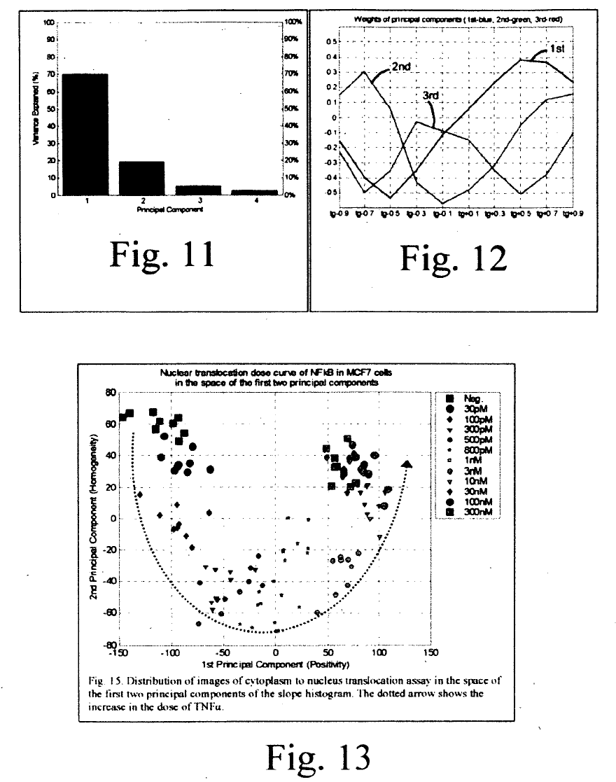

FIGS. 11-13 show results from a multidimensional PCA analysis of the data in FIG. 10. The multidimensional vector in this example is the histogram of distribution of slopes in cells, as shown in FIG. 11. Features are bins in the histogram and cases are doses and, possibly, replicas at each dose. Principal components are a non-correlated set of vectors, which are linear combinations of the original vector set. Depending on the nature of the data set, the first few principal components may explain the majority of variation in it. For example, here the first two principal components explain almost 90% of the variation, so it is reasonable to reduce the dimensionality of the data set from ten to two. The meaning is assigned to the principal components by analyzing their weights on the original features, as shown in FIG. 12. In this example, the first principal component can be interpreted as positivity of the translocation, since the weights of positive slopes are positive and the weights of negative slopes are negative. The second principal component can be interpreted as homogeneity, since both highly positive and highly negative slopes have positive weights and the slopes around zero have negative weights. The nuclear translocation dose curve, or distribution of points (images of cytoplasm to nucleus translocation assay) for NFκB in MCF7 cells, may be plotted in the space of the first two principal components of the slope histogram, as shown in FIG. 13.

1.10 Assay and Algorithm Quality Measures for Cell-Imaging Assays

In cellular imaging assays, the measure (or measures) used to characterize the assay may be far removed from the signal registered by the camera. Moreover, different algorithms may produce different assay measures on the same image. This is especially acute for redistribution (e.g., nuclear translocation) assays, where the total intensity may not change, and where the assay result may depend more on the algorithm than on the raw image. To decide which resolution is minimally acceptable for a given assay and algorithm, we analyze the same well area at different optical magnifications or/and the same set of images at different interpolated magnifications. In a similar manner, the effect of the cell number is analyzed by comparing measures from images of different size. To compare results, we use quality metrics discussed here.

The quality of assays, such as high-throughput screening assays, may be evaluated by a statistical parameter that depends on the dynamic range and variability of the assay, such as the z-factor [9]:

$$Z = 1 - 3 ( \frac{{SD}_{pos} + {SD}_{neg}}{\vert {M}_{pos} - {M}_{neg} \vert} )$$

Here, SD is standard deviation, M is mean, and pos and neg are the two extreme states of the assay, which define its dynamic range. The Z-factor ranges from −∞ to 1. For cell-based assays, z-factors above 0.5 are considered good. The z-factor has proved to be very useful for capturing and comparing variability caused by assay biology and by instrumentation (e.g., pipetting). Cell assays based on imaging introduce several new variables: imaging resolution, size of the imaged area, and the data extraction algorithm. Size of the imaged area is a variable because usually less than the whole system (e.g., less than the whole microplate well) is imaged and analyzed. Having a quality measure, like the z-factor, allows us to optimize variables that are under our control, e.g., find the best data extraction algorithm. Here, we will deal with specific cell image analysis algorithms and will use the quality measure to optimize image resolution and size.

Cellular imaging assays may lead us to reconsider the quality measure itself, in addition to introducing new variables. Assay measures derived from an image may be computationally very complex. For example, they may contain operations that have the effect of saturating the values from the positive and negative states of the assay, artificially reducing variability. This may happen unintentionally and even without being realized. Moreover, if the values of the assay for its positive and negative states do not overlap (and if they do it may not be a very useful assay), the z-factor can be manipulated intentionally, by applying a mathematical transformation that maps all positive values into a single value and all negative values into another single value. One way of dealing with this is the use in the quality measure of a dose-dependent sequence of assay states (dose-curve), with doses being close enough to each other, so that artificial manipulation would be impossible. This leads to the following measure, which we refer to as the “v-factor”:

$$V = 1 - 6 ( \frac{{SD}_{of\_fit}}{\vert {M}_{pos} - {M}_{neg} \vert} ) , \\ where$$

$${SD}_{of\_fit} = \sqrt{{{\sum }_{1}}^{n} \frac{{( {f}_{exp} - {f}_{mod} )}^{2}}{n}} .$$

Here, fₑₓₚ and f_{mod} are experimental and model values of the assay measure at a given concentration, respectively, and n is the number of experimental points in the dose curve.

The v-factor reverts to z-factor if there are only two dose points. The model may be chosen depending on the nature of response, with logistic curves often being the natural choice. Alternatively, in some cases, no specific model is used, and the average of several replicas is used as f_{mod} in the above equation. Then, the v-factor is given by the formula:

$$V = 1 - 6 ( \frac{Average\_SD}{\vert {M}_{pos} - {M}_{neg} \vert} )$$

The v-factor is less susceptible to saturation artifacts caused by computation than z-factor. There is also another subtle difference. Standard deviation in the middle of the dose-response curve often is larger than the standard deviation at the extremes. This is because the maximal point on the curve often is determined at saturating concentration, and so any dispensing error has little effect on the response. The minimal point usually is zero concentration, and it also avoids dispensing errors. In contrast, the effect of volume errors has its maximal effect in the middle of the dose-response curve. Thus, for at least these reasons, taking the whole curve into account may provide a more realistic measure of the assay data quality.
1.11 Dose dependency. Image Size and Magnification Dependency.

The average value of the individual cell slopes may be used as an assay parameter; for example, to characterize data from a well.

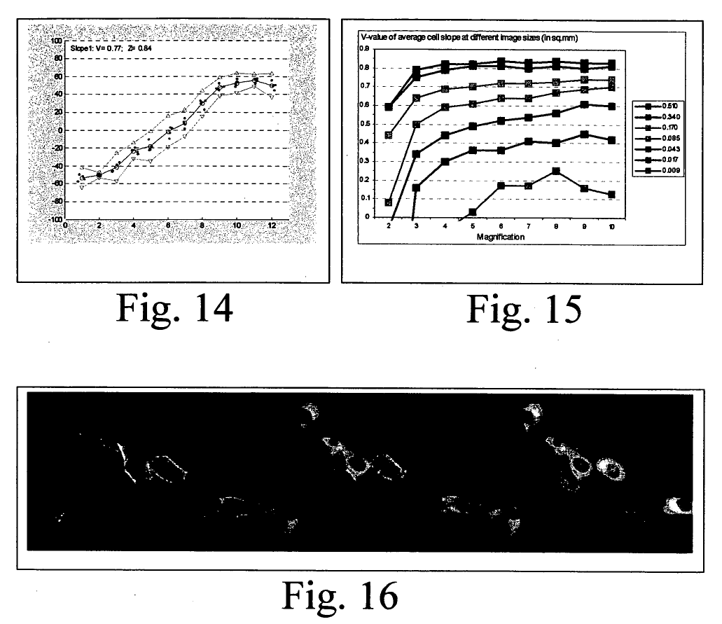

FIG. 14 shows a nuclear translocation dose curve, used to evaluate the suitability of average cell slope as an assay parameter. Data were collected from a dose-dependent set of images, such as those in FIG. 2. Here, average cell slope (vertical axis) is plotted as a function of TNFA concentration (horizontal axis). The corresponding v-factor is 0.77.

FIG. 15 shows the v-factor (vertical axis) for nuclear translocation measure as a function of interpolated magnification (horizontal axis) for different image sizes (reported in square millimeters). Specifically, the behavior of this algorithm was studied as a function of (1) interpolated image magnification, from the original 1 × magnification down to 2× magnification, and (2) image size, from 0.510 mm² down to 0.009 mm². The v-factor was used as a measure of quality. Image interpolation was done by the bilinear method. To study image-size dependency, the original image for each point in the curve was divided into fragment images of smaller sizes. Next, each of the smaller images was used to produce the translocation measure, and these measures were used in the formula for v-factor. The results for these different analyses, shown in FIG. 15, show that the algorithm reaches a plateau of v-factor around 0.8 at magnifications of 4× or greater and image sizes of 0.34 mm² or greater.

The average cell slope algorithm may have several desirable features: (1) it does not require segmentation into subcellular compartments; (2) it scales well with magnification; (3) it requires no user-settable parameters; (4) it is not sensitive to the overall intensity of the image, or to variations in intensity among cells; (5) it is based on a model that allows us to test the effects of disturbances (e.g., noise, irregular shape, etc.) and find a stable measure; and (6) it can be used globally and/or at the level of individual cells.

1.12 Optimization of Parameters. Selection of Best Measures.

The quality measures described in Section 1.10 can be applied if there are at least two points (and corresponding images) that can be used as a reference for a larger group of images that must be analyzed. An example of this arrangement would be a plate with some wells serving as positive and negative controls and other wells serving as test wells. In dose curve experiments, the whole curve can be used to calculate quality. Once the quality measure and the sample to which it is applied are established, one can pose a problem of optimizing parameters to achieve the highest possible quality. Similarly, if several measures with the same biological meaning are returned by an algorithm (e.g., slope1 or slope3; individual slope or global slope), the best of them can be chosen on the basis of quality.

The measures of translocation described here do not have any truly user defined parameters, at least in the same sense as the width of the ring¹ is a user parameter. However, there are some parameters built into the algorithm that may benefit from or need adjustment for a new cell type or specifics of staining, e.g., parameters controlling detection of markers and watersheds as described in Section 1.5. Suitable methods of optimization are well-known in the art [13].

Practical applications of optimization may vary. Positive and negative controls may exist on every plate, once for a group of plates, or (in some cases) be calculated rather than measured. In dose curve experiments, each curve can be optimized individually, or optimization may occur for a designated control curve, among others.

### Example 2

### Membrane to Cytoplasm Translocation Assay

This example describes another exemplary embodiment of the present teachings: a membrane-to-cytoplasm (or cytoplasm-to-membrane) translocation assay. In this assay, labeled moieties such as proteins translocate from the plasma membrane to the cytoplasm of the cell.

FIG. 16 shows a kinetic series of images of GFP-labeled live cells. Here, the left panel is a negative state (no translocation), the middle panel is an intermediate state (some translocation), and the right panel is a positive state (significant translocation).

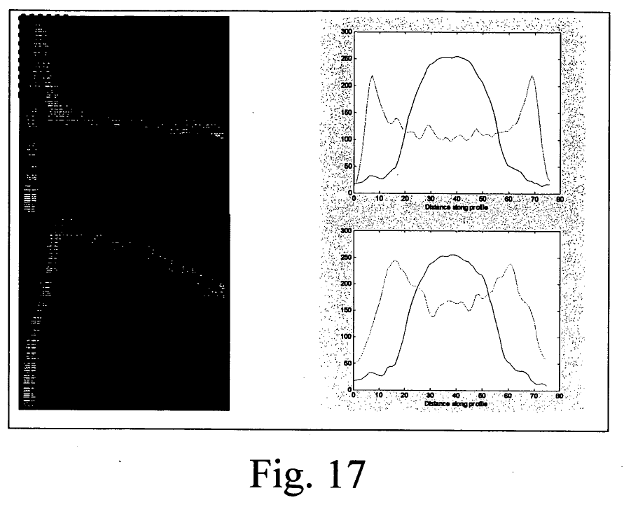

FIG. 17 shows a model of the joint distribution of signal and counter stains in membrane to cytoplasm translocation. Here, the top panels show membrane localization, and the bottom panels show cytoplasmic localization. The counterstain has a high level of staining in the nucleus and a low, but non-zero, level of staining the cytoplasm.

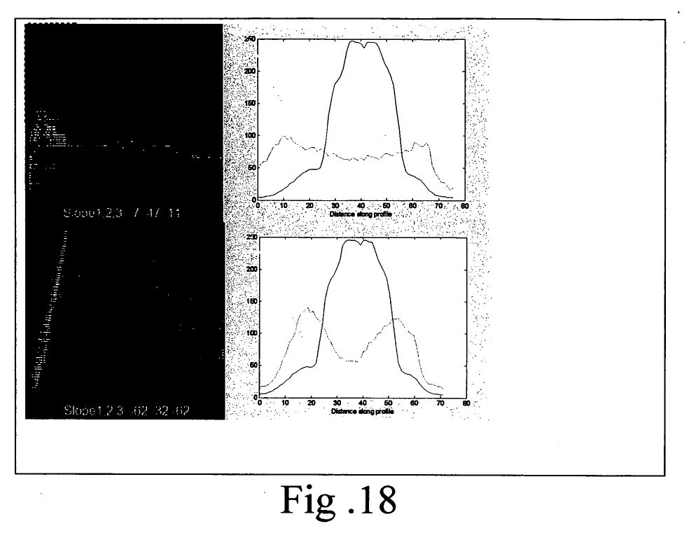

FIG. 18 shows distributions from real cells. Here, the top and bottom panels show membrane and cytoplasmic localization, respectively, as in FIG. 17. The same.measure of slope defined above can be used to characterize membrane to cytoplasm translocation. In addition, the histograms suggest the use of measures based on the points above the continuation of the slope segment to the Y-axis as shown by dotted rectangle in FIGS. 17 and 18.

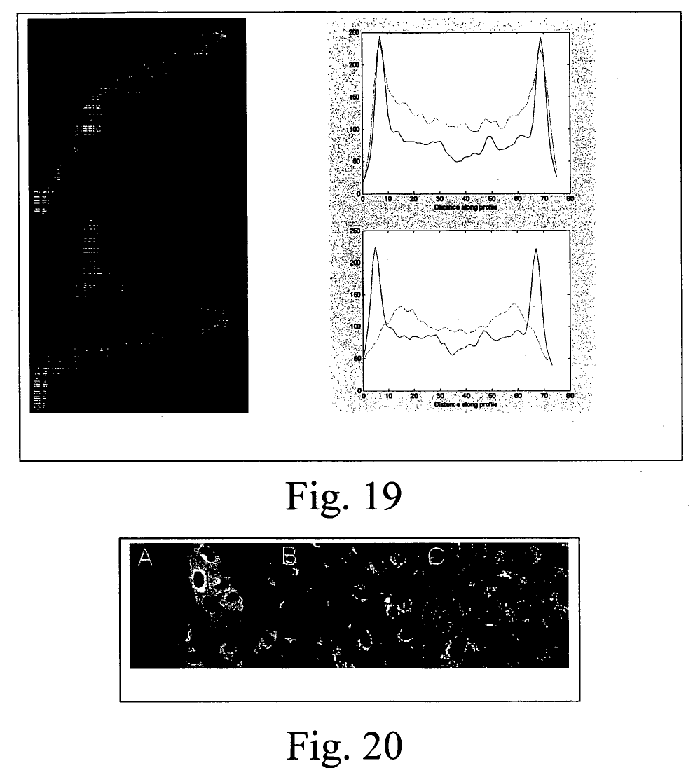

FIG. 19 shows a model of the joint distribution of signal and counterstains, where the counterstain used in the assay is not a nuclear stain, but a membrane stain. The membrane localization case can be characterized by a single slope, but the cytoplasmic localization uses two slope parameters.

More generally, the original histogram, which has 256*256 bins, can be divided in a coarser grid, as shown in FIG. 17. The size of bins can be chosen to provide a reasonable number of observations in each bin. Then, each 2-D histogram becomes a vector in the N-dimensional space, where N-is the number of bins. This allows treating the problem as a pattern recognition problem and using all the available arsenal of methods⁸.

### Example 3

### Diffuse to Granular Reorganization Assay

3.1. Background

Cellular components may rearrange from diffuse to granular sub-cellular patterns (or vice versa) in response to stimuli, such as treatment of cells with modulators. For example, proteins may be recruited to (and/or move to) sub-cytoplasmic domains (e.g., vesicles) or to sub-nuclear domains (e.g., PML bodies) in response to treatment with appropriate ligands. Accordingly, systems (including methods, algorithms, and apparatus) are needed to measure changes in the diffuseness of a reporter in, on, or about cells under various test conditions, such as exposure to a plurality of modulators of unknown effect in a screening assay.

3.2 Receptor Activation (Transfluor®)) Assay

The Transfluor® assay (commercialized by Xsira Pharmaceuticals™) is used to measure activity of G-protein coupled receptors (GPCRs). This assay employs green fluorescent protein (GFP) fused to β-arrestin as a reporter. The basis of the assay is to measure the sub-cellular localization of this fusion protein, which changes depending on receptor activity. In particular, the fusion protein changes from a diffuse cytoplasmic localization to a granular cytoplasmic (and/or membrane-associated) distribution upon receptor activation (e.g., ligand binding). Since β-arrestin is involved in the regulation of many GPCRs, it is thought of as a general assay, that is, one assay can serve to measure activity from different classes of GPCRs.

Receptor internalization in the Transfluor® assay causes images to exhibit a more granular distribution for the reporter. In particular, the reporter becomes distributed less uniformly within cells, to form “spots” or “dots” of concentrated reporter signal. Examples of Transfluor images are shown in FIG. 20. The images were collected using an objective magnification of 10× with 2*2 binning. Panel A shows “negative” cells without GPCR activation and exhibiting a diffuse distribution of the reporter. Panel B shows “intermediate” cells with partially activated GPCR. Panel C shows “positive” cells with fully activated GPCR and exhibiting a granular distribution of the reporter.

3.3 Methods of Analyzing Transfluor® Images

The present teachings provide a method for analyzing Transfluor® images. In some examples, the method may formalize the intuitive notion of granularity in a simple measure. For example, the method may employ the concept known in mathematical morphology as size distribution [11], granulometry [15], pattern spectrum [14], or granular spectrum [17]. A distribution is produced by a series of openings of the original image with structuring elements of increasing size. In the erosion step, the value of each pixel is set to a value corresponding to the minimum value of its surrounding pixels (e.g., the four pixels at its corners or sides, or the eight pixels completely surrounding the pixel, among others). In the dilation step, the value of each pixel is set to a value corresponding to the maximum value of its surrounding pixels. Each opening may include one or more successive erosion steps followed by one or more successive dilation steps. The number of erosion (and dilation) steps determines the size of the opening (and the size of the structuring element). For example, an opening of size “one” is produced by a single erosion and dilation step, an opening of size “two” by two erosion steps followed by two dilation steps, and so on. After each opening the volume of the resultant opened image is calculated as the sum of all pixels.

FIG. 21 shows how openings of increasing size affect images with different granularity. Brightness profiles are shown in panels A-C taken through cells (indicated in each panel inset by a line through cells). The three panels from left to right show negative, intermediate, and positive states of the Transfluor® assay. The graphs in each panel show brightness profiles for the original image (top profile), the image opened by a structuring element of size 1 (middle profile), and the image opened by a structuring element of size 4 (bottom profile).

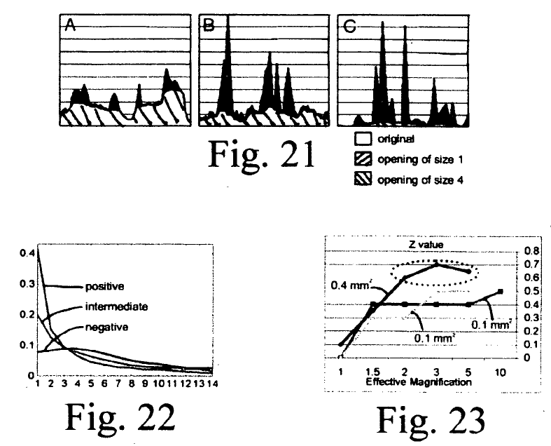

The difference in volume of the image, opened with different opening sizes, is the granular spectrum, given by the formula:
G(n)=V(γₙ₋₁(X))−V(γₙ(X))
Where X is the image, n is the opening size, also referred to as thickness, G(n) is the granular spectrum at the n-th opening, γₙ(X) is the n-th opening of image X, V(X) is the volume (sum of pixels values) of image X. Granular spectra for the negative, intermediate, and positive states of the assay are shown in FIG. 22, with the size of the opening (x-axis) plotted against the fraction of the image volume at this opening (y-axis). To characterize the different states of the assay we introduced a measure called relative granularity, given by the following formula:
RG=G(T1)/G(T2),
Where RG is relative granularity, T1 is the thickness most characteristic of the granular (positive) state of the assay, T2 is the thickness most characteristic of the diffuse (negative) state of the assay. T1 and T2 do not have to be single values but can be ranges of thickness, in which case the average of the granular spectral values is taken. Use of area opening [16] instead of opening to produce the granular spectrum may be beneficial.

To study the effects of the magnification and image sizes on relative granularity we used z-values because a detailed dose curve was not available. Two sets of images were used for experiments: one set for the positive state and one for the negative state. In each set one image was acquired using a 10× objective and one using a 20× objective, both with 2 by 2 binning; so in terms of spatial resolution we refer to them here as 5× and 10× magnifications. This has the benefit of making the plots comparable with other assays described. The image at 20× corresponds to the middle quarter of the 10× image. In addition we used an image that is the middle quarter of the 10× image. Each of the three images was divided in four fragments and an assay measure, relative granularity, was calculated for each of the fragments for the negative and positive state. Z values were then calculated using positive and negative sets. FIG. 23 shows the window of good assay performance (indicated with a dashed ellipse) at magnifications of 2× and above and an image size of 0.4 mm².

The algorithm presented above may have several desirable features: (1) requires no segmentation, (2) scales well with magnification, (3) has clear biological meaning, (4) does not require setting of any user parameters, and (5) is not sensitive to overall image intensity, which can be caused by differences in camera setting.

### Example 4

### Exemplary Embodiments

This example describes selected embodiments of the present teachings, presented as a series of numbered paragraphs.

1. A method of calculating a measure of the joint distribution of reporters in biological cells, comprising: (A) providing at least two reporters that can be visualized in cells; (B) acquiring digital images of the reporters in cells; and (C) using an at least two-dimensional distribution of values of the images of reporters to calculate a measure characteristic of a condition of the cells.

2. The method of paragraph 1, wherein there are N reporters, and wherein the step of using includes a step of forming at least one histogram selected from the group consisting of an N-dimensional histogram of values of reporters in the set of images of the same objects, a number of 2-dimensional histograms of the values of reporters in the set of images of the same objects, and a number of histograms of dimensionality between 2 and N of the values of reporters in the set of images of the same objects.

3. The method of paragraph 1, wherein the step of using includes a step of normalizing (locally equalizing) intensities of at least one of the reporter images.

4. The method of paragraph 1, wherein the step of using is performed on an individual cell-by-cell basis.

5. The method of paragraph 1, wherein the step of using is performed for a subset of cells in the image (can be individual by cells or for the subgroup as a whole).

6. The method of paragraph 1, wherein the step of using is performed for the whole image without identifying individual cells.

7. The method of paragraph 1, wherein the step of using includes a step of removing artifacts from the image(s).

8. The method of paragraph 2, wherein the step of using further includes fitting a model to the N-dimensional histogram, and wherein the measures are parameters of the model.

9. The method of paragraph 1, wherein the first reporter is associated with a cell compartment and the second reporter is associated with a protein (or other substance) that can change its localization from one cell compartment to another cell compartment under experimental conditions.

10. The method of paragraph 1, wherein the first reporter is associated with the nucleus and the second reporter is associated with a protein (or other substance) that can change its localization from cytoplasm to nucleus, or nucleus to cytoplasm, under experimental conditions.

11. The method of paragraph 1, wherein the first reporter is associated with the nucleus and the second reporter is associated with a protein (or other substance) that can change its localization from cell membrane to cytoplasm, or cytoplasm to cell membrane, under experimental conditions.

12. The method of paragraph 1, wherein the first reporter is associated with the cell membrane and the second reporter is associated with a protein (or other substance) that can change its localization from cell membrane to cytoplasm, or cytoplasm to cell membrane, under experimental conditions.

13. The method of paragraphs 8 and 10, wherein the model is a straight line segment of variable length approximating the right side of the distribution of the translocating protein reporter versus nuclear reporter (e.g., as shown in FIG. 20), and wherein the measure is the slope of this line.

14. The method of paragraphs 8 and 11, wherein the model is based on a straight line segment of variable length approximating the right side of the distribution of the translocating protein reporter versus nuclear reporter (e.g., as shown in FIG. 26), and wherein the measure is a statistic of a subset of points in the distribution (e.g., as shown in FIG. 26).

15. The method of paragraphs 8 and 12, wherein the model is based on a straight line segment of variable length approximating the right side of the distribution of the translocating protein reporter versus membrane reporter (e.g., as shown in FIG. 28).

16. The method of paragraph 2, wherein the N-dimensional histogram is viewed as an M-dimensional vector (M is the total number of bins in such histogram), wherein each cell (or a cluster of cells, or the whole image) is viewed as a point in the M-dimensional space, and wherein cells are analyzed using a method of pattern recognition.

17. The method of paragraph 16, wherein such method of pattern recognition is the classification of cells into predefined classes, and wherein the measures are the degree of similarity to such class and the class name.

18. The method of paragraph 1, wherein reporter images are preprocessed to deemphasize or correct some undesirable feature(s) (e.g., to fill holes due to nucleoli) or to emphasize some desirable feature(s).

19. The method of paragraph 4, wherein the population of cells is characterized by a statistical measure of position or by a statistical measure of variation.

20. The method of paragraph 19, wherein the measure of position is chosen from the group consisting of average, median, mode, etc.; and wherein the measure of variation is chosen from the group consisting of standard deviation, median deviation around median, etc.

21. The method of paragraph 4, wherein the population of cells is characterized by principal component analysis (PCA) of the histograms of distributions of the individual cell measures.

22. The method of paragraph 1, wherein measures are nominal (classification) measures of cell state, e.g., phase of cell cycle.

23. The method of paragraph 1, wherein the step of acquiring digital images is performed simultaneously for at least two different reporters.

24. The method of paragraph 1, wherein the step of acquiring digital images is performed sequentially for at least two different reporters.

25. The method of paragraph 1, wherein the measure is at low magnification, e.g. ≦2× objective (˜≧5 μm/pixel).

26. A method of calculating a measure of the joint distribution of reporters in biological cells, comprising: (A) providing at least two reporters that can be visualized in cells; (B) acquiring digital images of the reporters in cells in at least two test conditions; (C) using an at least 2-dimensional distribution of reporter values to calculate measures characteristic of a cell condition; and (D) providing a quality metric calculated on cellular measures in the at least two test conditions.

27. The method of paragraph 26, wherein the step of using includes an image analysis method dependent on a set of numerical parameters.

28. The method of paragraph 27, wherein the values of numerical parameters are chosen to optimize the quality metric calculated on cellular measures in the at least two test conditions.

29. The method of paragraph 26, wherein the step of using includes at least two methods of calculating cellular measures and the selection of the method that gives the best quality metric on the at least two test conditions.

30. The method of paragraph 26, wherein the step of using includes the step of selecting image subsets that give the best quality metric (e.g. systematically best camera field in the well or systematically best area in a camera field—mostly for reasons of focusing).

31. The method of any of paragraphs 28, 29, and 30, wherein the selection (optimization) is performed on one set of at least two test conditions and applied to other test conditions.

32. The method of paragraph 26, wherein the test conditions are different concentrations of a reagent.

33. The method of paragraph 32, wherein the reagent is a candidate drug compound.

34. The method of paragraph 26, wherein the test conditions are different time points of a certain process.

35. A method of partitioning an image with biological cells into fragments containing individual cells or clusters of cells, comprising performing a watershed transformation on an image that is a combination of images of at least two reporters.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

### REFERENCES

1. U.S. Pat. No. 5,989,835, entitled “System for cell-based screening.”
2. PCT Publication No. WO 03/078965, entitled “System and method for automatic color segmentation and minimum significant response for measurement of fractional localized intensity of cellular compartments.”
3. U.S. Publication No. 2003/0059093, entitled “Methods for determining the organization of a cellular component of interest.”
4. U.S. Publication No. 2003/0202689, entitled “Ray-based image analysis for biological specimens.”
5. S. Beucher and F. Meyer, “The Morphological Approach to Segmentation: The Watershed Transformation” in: Mathematical Morphology in Image Processing, E. R. Dougherty—Ed., pp. 433-481, Marcel Dekker, New York, 1993.
6. L. Vincent and P. Soille, “Watersheds in Digital Spaces: An Efficient Algorithm Based on Immersion Simulations”, IEEE Transactions of Pattern Analysis and Machine Intelligence, 13, No. 6, pp. 583-598, 1991.
7. I. Ravkin, V. Temov, A. Nelson, M. Zarowitz, M. Hoopes, Y. Verhovsky, G. Ascue, S. Goldbard, O. Beske, B. Bhagwat, and H. Marciniak, “Multiplexed high-throughput image cytometry using encoded carriers”, Proc. SPIE Vol. 5322, pp. 52-63, 2004 (Imaging, Manipulation, and Analysis of Biomolecules, Cells, and Tissues 11; D. Nicolau, J. Enderlein, R. Leif, and D. Farkas; Eds.)
8. R. Duda, P. Hart, and D. Stork, Pattern Classification, Wiley-Interscience, 2nd Ed., 2000.
9. J. Zhang, T. Chung, and K Oldenburg, “A simple statistical parameter for use in evaluation and validation of high throughput screening assays,” J. Biomol. Screening 4: pp. 67-73, 1999.
10. Image Processing Toolbox, The MathWorks, Inc. http://www.mathworks.com/products/image/.
11. J. Serra, Image Analysis and Mathematical Morphology, Vol. 1. Academic Press, London, 1989.
12. I. Jolliffe, Principal Component Analysis, Springer-Verlag, 2nd Ed., 2002.
13. R. Fletcher, Practical Methods of Optimization, Wiley, 2nd Ed., 2000.
14. P. Maragos, “Pattern spectrum and multiscale shape representation”, IEEE Transactions on Pattern Analysis and Machine Intelligence, 11, N 7, pp. 701-716, 1989.
15. L. Vincent, “Granulometries and Opening Trees,” Fundamenta Informaticae, 41, No. 1-2, pp. 57-90, IOS Press, 2000.
16. L. Vincent, “Morphological Area Opening and Closing for Grayscale Images,” Proc. NATO Shape in Picture Workshop, Driebergen, The Netherlands, pp. 197-208, 1992.
17. I. Ravkin and V. Temov, “Bit representation techniques and image processing,” Applied Informatics, v.14, pp. 41-90, Finances and Statistics, Moskow, 1988 (in Russian).

1. A method of calculating a measure of the joint distribution of reporters in biological cells, comprising:
providing at least two reporters that can be visualized in cells;
acquiring digital images of the reporters in cells; and
using an at least two-dimensional distribution of values of the images of reporters to calculate a measure characteristic of a condition of the cells.

2. The method of claim 1, wherein there are N reporters, and wherein the step of using includes a step of forming at least one histogram selected from the group consisting of an N-dimensional histogram of values of reporters in the set of images of the same objects, a number of 2-dimensional histograms of the values of reporters in the set of images of the same objects, and a number of histograms of dimensionality between 2 and N of the values of reporters in the set of images of the same objects.

3. The method of claim 1, wherein the step of using includes a step of normalizing intensities of at least one of the reporter images.

4. The method of claim 1, wherein the step of using is performed on an individual cell-by-cell basis.

5. The method of claim 1, wherein the step of using is performed without identifying individual cells.

6. The method of claim 1, wherein the step of using includes a step of removing artifacts from the image(s).

7. The method of claim 2, wherein the step of using further includes fitting a model to the N-dimensional histogram, and wherein the measures are parameters of the model.

8. The method of claim 1, wherein the first reporter is associated with a cell compartment and the second reporter is associated with a protein that can change its localization from one cell compartment to another cell compartment under experimental conditions.

9. The method of claim 1, wherein the first reporter is associated with the nucleus and the second reporter is associated with a protein that can change its localization from cytoplasm to nucleus, or nucleus to cytoplasm, under experimental conditions.

10. The method of claim 9, wherein there are N reporters, wherein the step of using includes a step of forming at least one histogram selected from the group consisting of an N-dimensional histogram of values of reporters in the set of images of the same objects, a number of 2-dimensional histograms of the values of reporters in the set of images of the same objects, and a number of histograms of dimensionality between 2 and N of the values of reporters in the set of images of the same objects, wherein the step of using further includes fitting a model to the N-dimensional histogram, wherein the measures are parameters of the model, wherein the model is a straight line segment of variable length approximating the right side of the distribution of the translocating protein reporter versus nuclear reporter, and wherein the measure is the slope of this line.

11. The method of claim 2, wherein the N-dimensional histogram is viewed as an M-dimensional vector (M is the total number of bins in such histogram), wherein each cell (or a cluster of cells, or the whole image) is viewed as a point in the M-dimensional space, and wherein cells are analyzed using a method of pattern recognition.

12. The method of claim 11, wherein such method of pattern recognition is the classification of cells into predefined classes, and wherein the measures are the degree of similarity to such class and the class name.

13. The method of claim 4, wherein the population of cells is characterized by a statistical measure of position or by a statistical measure of variation.

14. The method of claim 4, wherein the population of cells is characterized by principal component analysis (PCA) of the histograms of distributions of the individual cell measures.

15. A method of calculating a measure of the joint distribution of reporters in biological cells, comprising:
providing at least two reporters that can be visualized in cells;
acquiring digital images of the reporters in cells in at least two test conditions;
using an at least 2-dimensional distribution of reporter values to calculate measures characteristic of a condition of the cells; and
providing a quality metric calculated on cellular measures in the at least two test conditions.

16. The method of claim 15, wherein the step of using includes an image analysis method dependent on a set of numerical parameters.

17. The method of claim 15, wherein the step of using includes the step of selecting image subsets that give the best quality metric.

18. The method of claim 15, wherein the test conditions are different concentrations of a reagent.

19. The method of claim 18, wherein the reagent is a candidate drug compound.

20. The method of claim 15, wherein the test conditions are different time points of a certain process.
